(19) United States
(12) Patent Application Publication
(10) Pub. No.: US 2010/0222725 A1
(43) Pub. Date: Sep. 2, 2010

(54) PROTECTIVE LIMB DRAPE

(76) Inventor: Jon Munzel, San Francisco, CA (US)

Correspondence Address: GIFFORD, KRASS, SPRINKLE,ANDERSON & CITKOWSKI, P.C, PO BOX 7021, TROY, MI, 48007-7021, US

(21) Appl. No.: 12/714,804

(22) Filed: Mar. 1, 2010

Related U.S. Application Data
(60) Provisional application No. 61/156,156, filed on Feb. 27, 2009.

(51) Int. Cl. A61F 5/00

(52) U.S. Cl. 602/3

(57) ABSTRACT

A protective limb drape is provided that includes a polymer sheet surrounding an aperture and having attachments along a polymer sheet edge to facilitate suspension of said polymer sheet edge to facilitate suspension of the polymer sheet from a generally horizontal shower curtain rod. An elastomeric ribbon is attached to the sheet to at least partially cover the aperture to define a portal sized to allow the limb to traverse the polymer sheet through the aperture. A flap is attached to a first side of the sheet along a flap edge to overlie the portal. A protective limb drape is also provided as detailed above including a second flap attached to an opposing side of the polymeric sheet with positional overlap with the first flap. Additional apertures defined by the polymeric sheet and a duplicate elastomeric ribbon are also optionally provided such that a single inventive drape is able to accommodate either an arm or a leg traversing the polymeric sheet.

### CROSS-REFERENCE TO RELATED APPLICATION

This application claims priority of U.S. Provisional Patent Application Ser. No. 61/156,156 filed Feb. 27, 2009, which is incorporated herein by reference.

### FIELD OF THE INVENTION

The present invention in general relates to a drape for protection of an injured limb and in particular to a drape mounted as a shower curtain.

### BACKGROUND OF THE INVENTION

To prevent complications, it is often required that an injured limb be kept dry. Casts, open deep wounds, and external orthopedic pins are exemplary of wounds that should be kept dry. As a result of the requirement that a limb (an arm or a leg) be kept dry, the maintenance of body hygiene is complicated. In many instances, these complications are dealt with simply by foregoing immersion bathing or showering in favor of a sponge bath. Unfortunately, water sponge baths are less than effective at maintaining body hygiene while alcoholic sponge baths tend to dry skin with prolonged usage. Immersion baths are problematic in that it is difficult to maintain balance while entering the tub and avoiding inadvertent wetting.

Showering with an injured limb poses particular difficulties in that a high pressure water spray and gravity fed water trickle pose two separate modes of wetting. In an attempt to allow an individual having a limb that needs to be maintained in a dry state to shower, numerous prior art attempts have been made to develop a waterproof cover with a seal around the limb that is also waterproof. Representative of these efforts are U.S. Pat. Nos. and Publications 3,785,374; 4,139,003; 4,406,281; 5,728,052; 6,210,352; 6,336,232; 7,198,608; 2005/0027227; and 2006/0276734. Existing limb covers have met with limited success owing to problems ranging from limiting blood circulation, difficulty of limb insertion, and sweating associated with limb enclosure.

Thus, there exists a need for a protective limb drape that is effective at keeping the limb dry, inexpensive, easy to use, and comfortable to use.

### SUMMARY OF THE INVENTION

A protective limb drape is provided that includes a polymer sheet surrounding an aperture and having attachments along a polymer sheet edge to facilitate suspension of said polymer sheet edge to facilitate suspension of the polymer sheet from a generally horizontal shower curtain rod. An elastomeric ribbon is attached to the sheet to at least partially cover the aperture to define a portal sized to allow the limb to traverse the polymer sheet through the aperture. A flap is attached to a first side of the sheet along a flap edge to overlie the portal. A protective limb drape is also provided as detailed above including a second flap attached to an opposing side of the polymeric sheet with positional overlap with the first flap. Additional apertures defined by the polymeric sheet and a duplicate elastomeric ribbon are also optionally provided such that a single inventive drape is able to accommodate either an arm or a leg traversing the polymeric sheet.

### BRIEF DESCRIPTION OF THE DRAWINGS

FIG. 1 is a perspective view of an inventive drape in the context of a shower curtain;

FIG. 2A is a front view of a portal formed by an elastomeric rubber partially covering an aperture in a polymeric sheet;

FIG. 2B is a magnified perspective view of a portal flap and drape of FIG. 1;

FIG. 3 is a cross-sectional view of an inventive drape showing opposing flaps covering an aperture therethrough; and

FIG. 4 is an exploded view of a limb drape module amenable to custom insertion in a shower curtain.

### DETAILED DESCRIPTION OF THE INVENTION

The present invention has utility as a drape that protects an injured limb from water exposure. The present invention uses an elastomeric-edged aperture to faun a seal between the limb and the remainder of the inventive drape thereby eliminating the need for the mitt-like or boot-like covers conventional to the art.

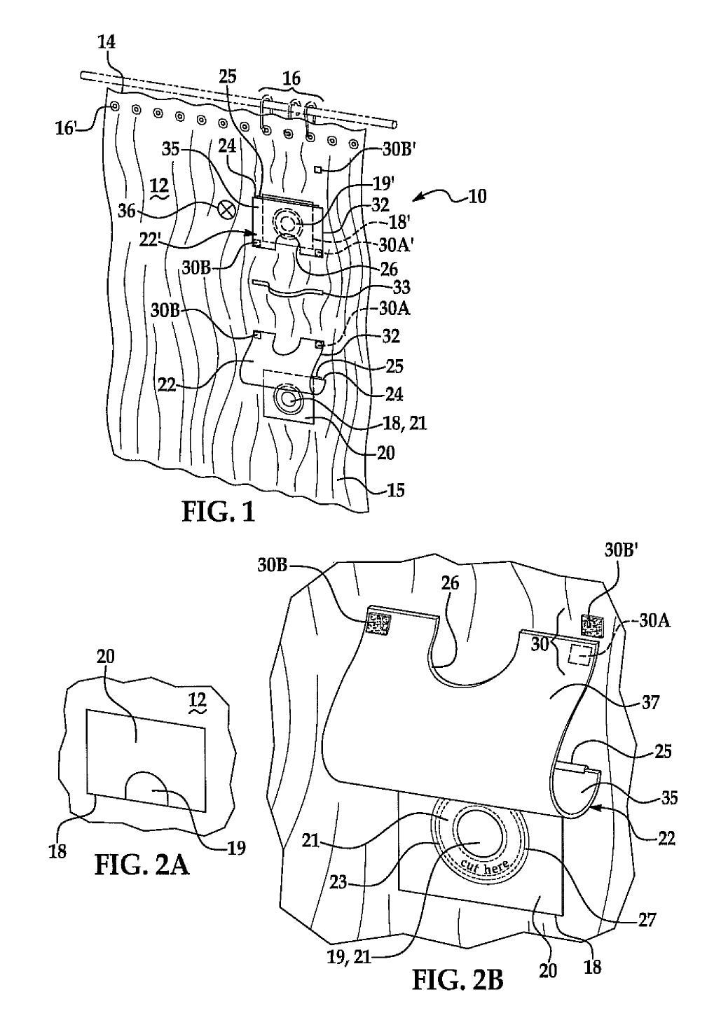

Referring now to FIGS. 1-3, an inventive drape is depicted generally at 10. The drape 10 is formed of a polymer sheet 12 that is impervious to water. Materials suitable for the formation of a polymeric sheet 12 illustratively include thermoplastics such as polyolefins, polyvinyl chlorides, polyesters, and polyurethanes; elastomers such as butyl rubber and latexes; and copolymers of the aforementioned. Preferably, the polymeric sheet 12 is flexible as defined by folding when stood upon edge under the weight of the polymeric sheet 12. Optionally, the polymeric sheet 12 has an edge 14 modified with securements 16 to facilitate suspension of the polymeric sheet 12 from a generally horizontal rod so as to allow an inventive drape 10 to function as a shower curtain. A securement 16 illustratively includes multiple apertures, material loops, or conventional shower curtain suspension hardware. Various securements 16 are accordingly depicted in the accompanying figures. Optionally, a second set of securements 16′ are provided vertically displaced from securements 16 so as to provide alternative height positioning of the inventive drape 10 and elements thereof. It is appreciated that the second set of securements 16′ are of a same type or different type relative to securements 16 in a given embodiment. By way of example, when securements 16 are apertures, securements 16′ are also apertures or material loops. An aperture 18 is defined by the polymeric sheet 12. Optionally, the surface of the polymeric sheet includes a handle 33 to facilitate casted limb insertion through aperture 18 for movement of a drape 10. A handle 33 is optionally formed from a strip of the same material from which the polymeric sheet 12 is formed being welded thereto to form a grippable loop.

An elastic ribbon 20 is attached to the polymeric sheet around at least a portion of the aperture 18 to reduce the area of the aperture 18 to form a portal 19 sized to permit transit of an injured limb therethrough. FIG. 2A depicts a ribbon 20 that only partially covers aperture 18. Preferably, the ribbon 20 covers the aperture 18 and has a through hole 21 defining portal 19, as shown in FIG. 2B at 19/21. Optionally, a rigid plastic ring 27 bounds and supports the ribbon 20 to define the portal 19 or the portal 19 is defined between the ribbon 20 and the ring 27. Materials from which the elastomeric ribbon 20 is formed illustratively include isoprene, epoxidized natural rubber, ethylene propylene diene M-class rubber (EPDM), latex, butyl rubber, and vinyl. An elastomeric ribbon 20 is appreciated to also constitute an elastomeric sheet secured to the polymeric sheet 12 overlapping the aperture 18, the elastomeric sheet having the through hole 21 folioed therethrough to define the portal 19 upon. To facilitate a large casted limb or a height differential between the limb and portal 19, optionally the elastomeric ribbon 20 is provided with at least one thinned region 21 in the form of an arc, circle, or other shape. The thinned region 21 forms a least resistant tear line to enlarge the area of portal 19. Alternatively, the elastomeric ribbon 20 has printed indicia 23 designating cut lines to enlarge the area of portal 19.

In operation, as a user passes an injured limb through the portal 19, the ribbon 20 is tensioned against the proximal user limb relative to the distal injury. It is appreciated that the resultant seal created about the portal 19 by the ribbon 20 relative to a proximal user limb by itself in some circumstances is insufficient to prevent a high pressure shower spray on a first side of a polymeric sheet 12 from passing through the seal so created in the portal 19 to wet the injured limb now present on the second external side of the polymeric sheet 12.

To protect the seal created between the ribbon 20 and a user proximal limb within the portal 19 from high pressure shower spray, a flap 22 is secured vertically along a flap edge 24 above the aperture 18 and connected to a first side 15 of the polymeric sheet 12. The flap 22 is dimensioned such that the portal 19 is covered by the flap 22 over the vertical-most 90% of the aperture area. Preferably, the flap 22 when extending fold-free along the polymeric sheet 12 completely covers the portal 19. More preferably, the flap 22 extends beyond the portal 19 to the maximal extent and has a slit or cutout 26 extending towards and preferably not overlapping the portal 19. Optionally, the flap 22 is secured to the plastic ring 27, as shown in FIG. 4. It is noted that the inclusion of a slit or cutout 26 facilitates the draping of a flap 22 over the proximal portion of an injured limb to effectively form a shield preventing high pressure water spray from a shower from contacting the seal between the ribbon and a proximal limb portion. In the context of an inventive drape being used in surgery, a direct linear path between the seal and the environment above a polymeric sheet through which an injured limb has been inserted is also blocked by the flap 22.

Optionally, at least one selectively openable fastener 30 is provided on an edge 32 of the flap 22 remote from the flap attachment 25. The fasteners 30 illustratively include snaps, contact adhesives, and hook-and-loop fasteners. Preferably, the attachment 25 between the flap 22 and the polymeric sheet 12 lacks a linear overlap with fastener 30 on an orthogonal axis relative to the attachment 25. As shown in particular detail in FIG. 2, a first portion 30A of a fastener 30 is secured to an outward face 35 of the flap 22. The complementary portion 30B of the fastener 30 is secured to the opposing inward face 37 of the flap 22 thereby shaping the flap 22 in a conical shape that facilitates engagement of a proximal limb. Optionally, a second complementary portion 30B′ is secured to the first side 15 of the polymeric sheet 12 to selectively hold the flap 22 in an open position by joining portions 30A and 30B′.

It is appreciated that an inventive drape 10 optionally includes a duplicate flap 22′ and arm portal 19′ (shown in ghost with overlapping flap 22′) to facilitate operation as a shower curtain for both casted arms or legs. As detailed above with portals 19 and 22, portals 19′ and 22′ are spaced to facilitate insertion of an injured arm. Like numerals used with respect to flap 22′ correspond to the meanings attributed to those of flap 22. Optionally, access opening 36 is provided in the polymeric sheet 12 positioned in proximity to the portal 19′ to allow the hand of an injured arm to be reinserted into contact with the first side of the polymeric sheet 12 depicted in FIG. 1 presented to the viewer. Optionally, an access opening 36 includes a sealed sleeve 37 formed as a tube, mitt, or glove to prevent wetting of the injured limb. It is noted that such an opening 36 facilitates use of an inventive drape 10 in the context of a shower curtain thereby allowing a user to grip a shampoo bottle or otherwise use the hand in the course of showering. The opening 36 optionally is bounded by an elastomeric diaphragm to inhibit water flow back through the hand opening 36 towards the injured limb.

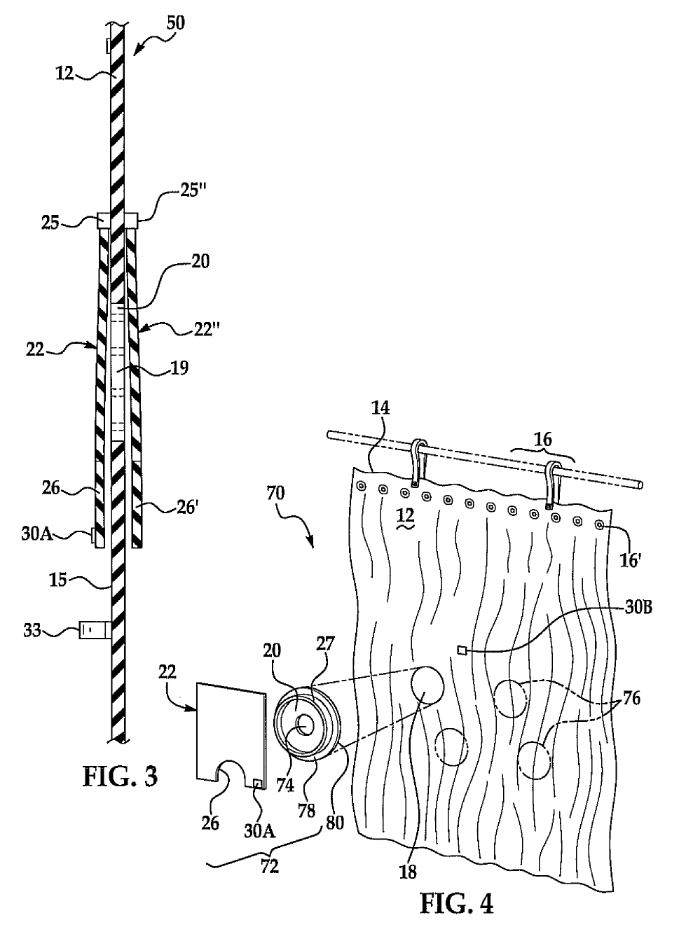

As shown in FIG. 3, an inventive drape 50 having a second flap 22″ otherwise the same as flap 22 is provided on the second side of the polymeric sheet 12 in opposition to the flap, where double primed numerals used with respect to FIG. 2 have the same meaning as used with respect to the base numeral as detailed above. It is appreciated that an opposing flap 22″ embodiment of an inventive drape 50 affords an additional barrier to water transit through aperture 18, as well as addressing inverted deployment of an inventive drape.

Referring now to FIG. 4, an inventive drape is shown generally at 70 where like reference numerals have the meanings ascribed thereto with respect to the aforementioned figures. The inventive drape 70 has a drape insert module 72 based on a plastic ring 27 supporting a portion of elastomeric ribbon 20 defining an aperture 74 and a flap 22. Other attributes depicted for the module 72 are optional. An aperture 74 is bound at least on one side by the ribbon 20 and, as shown, the aperture 74 is surrounded by the ribbon 20. A flap 22 is secured to the ring 27 to cover at least a portion of aperture 74 where the flap 22 is extended. Preferably, the extended flap 22 has a slit or cutout 26. The module 72 inserts into an aperture 18 formed in a polymeric sheet 12. The aperture 18 is optionally custom cut to the specifics of the limb enclosure and height of a user. Optionally, the sheet 12 has scores or thinned regions 76 to facilitate cutting sheet 12 to form aperture 18 in various positions, such as defined by regions 76. The module 72 is secured to the sheet 12 by conventional techniques including contact adhesives. A flange 78 with a pre-adhesed surface 80 that bonds to the sheet 12 bound aperture 18 is a representative joinder. Hook-and-loop fastener component 30B′ is optionally added to the sheet 12 after securement of the module 72.

Patent documents and publications mentioned in the specification are indicative of the levels of those skilled in the art to which the invention pertains. These documents and publications are incorporated herein by reference to the same extent as if each individual document or publication was specifically and individually incorporated herein by reference.

The foregoing description is illustrative of particular embodiments of the invention, but is not meant to be a limitation upon the practice thereof The following claims, including all equivalents thereof, are intended to define the scope of the invention.

1. A protective limb drape comprising:
a polymeric sheet surrounding an aperture and having attachments along a polymer sheet edge to facilitate suspension of said polymeric sheet from a generally horizontal shower rod curtain; and
an elastomeric ribbon attached to said sheet to at least partially cover the aperture to define a portal sized to receive a limb.

2. The drape of claim 1 wherein a first flap is attached to a first side of said sheet along a flap edge, said flap overlying the portal.

3. The drape of claim 1 wherein said elastomeric ribbon covers the aperture and has a through hole forming the portal.

4. The drape of claim 1 wherein the aperture is covered by said elastomeric ribbon, said ribbon having a through hole for receiving a limb, said elastomeric ribbon secured to a ring that is coupled to said polymer sheet.

5. The drape of claim 4 further comprising a ring flap secured to said ring and overlying the hole.

6. The drape of claim 2 wherein an attachment joins the flap edge to said sheet, said attachment not extending along a full linear extent of said first flap.

7. The drape of claim 2 further comprising a first fastener portion on an outward face of said first flap to said polymeric sheet remote from the flap edge, a complementary fastener portion on an inward face of said first flap and said first fastener portion and said complementary fastener portion positioned linearly from the unattached portion of the linear extent to shape said first flap in a conical shape upon joinder of said first fastener portion and said complementary fastener portion.

8. The drape of claim 2 further comprising at least one fastener selectively securing said first flap to said polymeric sheet.

9. The drape of claim 2 wherein said first flap extends beyond the opening and has an opposing flap edge relative to the flap edge, said first flap having a slit or a cutout extending from the opposing flap edge towards the flap edge.

10. The drape of claim 2 further comprising a second flap attached to an opposing side of said polymeric sheet relative to the first side with positional overlap with said first flap.

11. The drape of claim 1 further comprising a second set of securements displaced from said securements.

12. The drape of claim 1 further comprising at least one handle secured to said polymeric sheet.

13. The drape of claim 1 further comprising a thinned region in said elastomeric ribbon.

14. The drape of claim 1 further comprising printed indicia on said elastomeric ribbon denoting a cut line.

15. The drape of claim 1 wherein said polymeric sheet has an opening proximal to the aperture adapted to receive a user hand of an injured limb to be inserted therethrough.

16. The drape of claim 1 further comprising a second aperture in said sheet having a duplicate elastomeric ribbon attached to said sheet to at least partially cover the second aperture to define a second portal and a duplicate flap attached to said sheet overlying the second portal.

17. A protective limb drape comprising:
a polymeric sheet surrounding an aperture and having attachments along a polymer sheet edge to facilitate suspension of said polymeric sheet from a generally horizontal shower rod curtain;
an elastomeric ribbon attached to said sheet to at least partially cover the aperture to define a portal sized to receive a limb, wherein a first flap is attached to a first side of said sheet along a flap edge, said flap overlying the aperture; and
a second flap attached to an opposing side of said polymeric sheet relative to the first side with positional overlap with said first flap.

18. The drape of claim 17 wherein an attachment joins the flap edge to said sheet, said attachment not extending along a full linear extent of said first flap.

19. The drape of claim 17 wherein said first flap extends beyond the portal and has an opposing flap edge relative to the flap edge, said first flap having a slit or a cutout extending from the opposing flap edge towards the flap edge.

20. The drape of claim 17 further comprising at least one handle secured to said polymeric sheet.
